(19) United States
(12) Patent Application Publication
(10) Pub. No.: US 2008/0274162 A1
(43) Pub. Date: Nov. 6, 2008

(54) Method, composition, and delivery mode for treatment of prostatitis and other urogenital infections using a probiotic rectal suppository

(75) Inventors: Jeffrey Bryan Nessa, Shakopee, MN (US); Beth Ann Nessa, Shakopee, MN (US)

Correspondence Address: JEFFREY AND BETH NESSA, 1719 CROSSINGS BLVD., SHAKOPEE, MN, 55379, US

(73) Assignees: Jeffrey Nessa, Shakopee, MN (US); Beth Nessa, Shakopee, MN (US)

(21) Appl. No.: 11/744,308

(22) Filed: May 4, 2007

(51) Int. Cl. A61K 9/02; A61K 35/74; A61P 13/02

(52) U.S. Cl. 424/436; 424/93.45

(57) ABSTRACT

This invention is a probiotic composition using the delivery mode of a rectal suppository for the treatment and relief of symptoms of urogenital infections including prostatitis. This invention is unique in both mode of delivery and in enabling the treatment of such urogenital infections as prostatitis. The present invention discloses compositions, methodologies, and delivery mode for the utilization of probiotic organisms in therapeutic compositions for treatment and relief from the symptoms of urogenital infections by maintaining and restoring normal flora in humans. The composition includes one or more bacteria selected from the genus Lactobacillus. More specifically, the invention relates to the utilization of one or more species or strains of Lactobacillus producing bacteria for the control of urinary tract bacteria and pathogens, including antibiotic-resistant urinary tract pathogens, and their associated diseases by both a reduction in the rate of colonization of the infection and pathogens. The method for treatment and relief from symptoms includes administration via a rectal suppository with a safe and effective amount of the composition. The delivery mode for administering the probiotic is via a rectal suppository. The delivery through the rectum provides an optimum method of transmission of the treatment.

### FIELD OF THE INVENTION

This invention relates to bacterial compositions through rectal suppository mode for maintaining and restoring normal indigenous urogenital flora in a human. These compositions and methods may be employed to treat or prevent urogenital diseases and infections.

### BACKGROUND OF THE INVENTION

To understand the benefits of probiotic suppositories, a historical long term case study was conducted over six years. The invention was tested with many forms and compositions used, as discussed in the claims section. The subject was diagnosed with urogenital infections including chronic prostatitis with symptoms of an inflamed urethra and swollen prostate. The subject went through antibiotic s with minimal results and minimal benefits. The probiotic composition proved to be most effective when delivered by suppository mode. Case study many different forms and compositions used as discussed in claims. Research has concluded that suppositories prove to be an effective and superior mode of delivery to treat urogenital infections including, but not limited to, prostatitis.

In accordance with the present invention, rectally administered Lactobacillus species can colonize the human intestinal, genital and urinary tracts thereby inhibiting the colonization of urogenital pathogens. The rectally administered Lactobacillus species can also promote the restoration and maintenance of normal flora of the urogenital tract thereby preventing, treating and/or reducing the occurrence of infections caused by urogenital pathogens.

The urogenital bacteria inhibited and otherwise depleted by the Lactobacillus of the present invention include, but are not limited to, Escherichia coli, Klebsiella spp., Pseudomonas spp., Proteus spp., Providencia spp., Staphylococcus spp., Streptococcus spp., Bacteroides spp., Mobiluncus spp. Trichomonas spp. Fusobacterium spp., Enterococcus spp., Gardnerella spp. and yeast.

In the present patent, urogenital infections include all infections with symptoms and signs associated with infection of the urinary tract. This includes discharge from the urethra, pain during urination, urgency in urination, pain in the lower back, fever and chills. Specific examples of urogenital infections include, but are not limited to, urethritis, bacterial cystitis, interstitial cystitis, and prostatitis.

This invention relates to a method of delivering therapeutic agents to the urethra, bladder and related structures and a rectal suppository for use in delivering therapeutic agents thereto.

In the treatment of various ailments affecting the urethra, bladder and related structures, systemic delivery such as oral, subcutaneous, or transdermal methods, lose much of the efficiency and effectiveness of the treatment through dilution and dissipation. Additionally, taken orally, the treatment can also lose effectiveness from biochemical interactions between the therapeutic agent and unrelated systems. The rectal suppository delivery method locally applies the therapeutic agents in the treatment of urogenital infections by administering relatively high concentrations of the therapeutic agents to the affected areas.

While the local application of therapeutic agents permits the use of smaller dosages, and can avoid certain of the drawbacks associated with systemic delivery methods as mentioned hereinabove, a different set of problems arise with respect thereto. In the context of treating conditions affecting the urethra, bladder and related structures, for example, it is of primary importance that the local application of therapeutic agents be accomplished in a manner compatible with the anatomical structures involved. Accordingly, it is known to employ suppositories as delivery devices for therapeutic agents. Such suppositories are designed to be inserted into the urethra and to release therapeutic agents contained therein or applied thereon to the mucosal lining of the urethra. The release of therapeutic agents occurs upon liquefaction of the suppository which results from the transfer to the suppository of the body heat of the patient into whose urethra the suppository has been placed.

It is well known that indigenous, non-pathogenic bacteria predominate on epithelial cells and associated mucus in the healthy state, and that pathogenic organisms predominate in the stages leading to and during infections. The possibility that indigenous bacteria have a role in preventing infection has been postulated for many years, but few studies have been carried out to identify specific bacteria and their properties required for such an effect.

The results of recent studies discussed the important role accomplished by lactobacilli in keeping a normal bacterial equilibrium within the body for the prevention of genital infections caused by pathogenic organisms. It is well known that many lactobacilli inhabit the urogenital areas of the body. Lactobacilli present in the vagina can succumb to administration of these antibiotics. Unfortunately, pathogenic bacteria (staphylococci, for example) can acquire a resistance to antibiotics and thus make it difficult to treat these diseases by administering antibiotics.

In U.S. Pat. No. 6,180,100, Lactobacillus casei, Lactobacillus acidophilus, Lactobacillus plantarum, and Lactobacillus jensenii is used for treatment of urinary tract infections. In U.S. Pat. No. 5,176,911 and U.S. Pat. No. 6,277,370, lactobacilli is used for treatment of vaginal infections.

This invention relates to bacterial compositions and methods for maintaining and restoring normal indigenous urogenital flora in a human. These compositions and methods may be employed to treat or prevent urogenital diseases and infections.

### SUMMARY OF THE INVENTION

This invention is a probiotic composition using the delivery mode of a rectal suppository for the treatment and relief of symptoms of urogenital infections including prostatitis.

The present invention discloses compositions, methodologies, and delivery mode for the utilization of probiotic organisms in therapeutic compositions.

This includes the method and composition for treatment and relief from the symptoms of urogenital infections by maintaining and restoring normal flora in humans. The composition includes one or more bacteria selected from the genus Lactobacillus. More specifically, the invention relates to the utilization of one or more species or strains of Lactobacillus producing bacteria for the control of urinary tract bacteria and pathogens, including antibiotic-resistant urinary tract pathogens, and their associated diseases by both a reduction in the rate of colonization of the infection and pathogens.

The method for treatment and relief from symptoms includes administration via a rectal suppository with a safe and effective amount of the composition. The delivery mode for administering the probiotic is via a rectal suppository. The delivery through the rectum provides an optimum method of transmission of the treatment.

### DETAILED DESCRIPTION OF THE INVENTION

As used herein, the term “inhibit” to prevent the normal growth of an organism or the activity of an enzyme or protein.

By “pharmaceutically-acceptable carrier” as used herein is meant one or more compatible solid or liquid filler diluents, or encapsulating substances, in which compatible is defined as components of the composition capable of being commingled without interacting in a manner which would substantially decrease the pharmaceutical efficiency under ordinary use situations. Some examples of substances which can serve as pharmaceutical carriers are sugars, starches, cellulose and its derivatives, oils such as coconut oil, peanut oils, cotton seed oil, sesame oil, olive oil, and corn oil; as well as other non-toxic compatible substances used in pharmaceutical formulations. Lubricants can also be present.

By “safe and effective amount” as used herein is meant a concentration high enough to significantly-positively modify the condition being treated but low enough to avoid serious side effects (at a reasonable benefit/risk ratio), within the scope of sound medical judgment. A safe and effective amount of probiotic will vary with varying conditions such as, but not limited to, age and physical condition of the patient being treated, the specific infection or condition being treated, the duration of treatment, and the nature of infection.

The term “urogenital” as used herein, are intended to be synonymous and refer to male and female urinary tract system, and includes prostate infections and prostatitis, urinary tract infections, and bladder infections.

The term “cfu” as used herein, are intended to refer to its common technical meaning as number of microbial colony forming units.

The term “gastrointestinal” as used herein, are intended to be synonymous and refer to the oral cavity, esophagus, stomach, small intestines, large intestines, colon, anus and perianal region.

The composition can comprise a safe and effective amount of one or more of the aforementioned bacteria with a pharmaceutically acceptable carrier.

Some forms of the composition may comprise one or more bacteria in a jelly base, preferably a K-Y jelly base. Another application involves the preparation of a freeze-dried capsule comprising the composition of the present invention. Effective dosages may range from 10.sup.3 to 10.sup.13 cfu per daily dose and more preferably from 10.sup.5 to 10.sup.10 cfu/ml per daily dose. Typically effective dosages are in the range of 10.sup.9 cfu/ml.

The treatment method may vary according to the individual condition of the subject. For example, one regimen involves the subject taking a continuous self administered dose one or more times a day. Another regimen involves the subject self administering a single dose at least once per week on an on-going basis. Yet another regiment involves the subject self administering one or more doses for a period of 1 to 120 days.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been described, it is obvious to those skilled in the art that various other changes and modifications can be made without departing from the spirit and scope of the invention. It is therefore intended to cover in the appended claims all such changes and modifications that are within the scope of this invention.

1. A composition for treatment of urigenital infections by maintaining and restoring normal indigenous urogenital flora in a human comprising one or more bacteria selected from the genus Lactobacillus and a pharmaceutically acceptable carrier.

2. A The composition of claim 1, wherein said composition further comprises one or more bacteria selected from the group of Lactobicillus bacteria consisting of Lactobacillus acidophilus, Lactobacillus rhamnosus, Lactobacillus casei, Lactobacillus gasseri, Lactobacillus fermentum, Lactobacillus amylolyticus, Lactobacillus casei subs. pseudoplantarum, Lactobacillus brevis, Lactobacillus salivarius, Lactobacillus plantarum, Lactobacillus fermentum, Lactobacillus jensenii, Lactobacillus crispatus, Lactobacillus vaginalis, Lactobacillus mucosae, Lactobacillus paracasei, and Lactobacillus coleohominis.

3. The composition according to claim 1 wherein said composition is in the form of a rectal suppository. The suppository will include an applicator device or implement that facilitates the insertion of a rectal suppository. Non-limiting specific examples of such include any known hygienically designed applicator that is capable of rectal insertion.

4. The composition of claim 1, wherein said composition further comprises the second or more bacteria selected from the group of Bifidobacterium.

5. The composition of claim 2, wherein said composition further comprises the second or more bacteria selected from the group of Bifidobacterium consisting of wherein said second probiotic organism is selected from the group consisting of Bifidobacterium bifidum, Bifidobacterium breve, Bifidobacterium longum, Bifidobacterium lactis, and Bifidobacterium adolescentis.

6. The composition of claim 1, wherein said composition is comprised solely of bacteria consisting of Lactobacillus acidophilus.

7. The composition according to claim 1 where the pharmaceutically acceptable carrier is coconut oil, extra virgin coconut oil, or organic extra virgin coconut oil.

8. The composition according to claim 1 where the pharmaceutically acceptable carrier wherein said milk portion is yogurt.

9. The method of claim 8 wherein each bacteria is administered in a dose of from about 10.sup.3 to about 10.sup.13 cfu/ml.

10. The method of claim 8 wherein each bacteria is administered in a dose of from about 10.sup.5 to about 10.sup.10 cfu/ml.

11. The method of claim 8 wherein the bacteria administered as a rectal suppository, created in the form of 1 billon CFU/g of L. acidophilus, B. Bifidum, B. longum, L. rhamnosus, with 12.5 mg of Fructooligosaccharide (FOS), and encapsulated in glycerin with the carrier of coconut oil.

12. The method of claim 11 wherein the bacteria administered also includes 10 mg L. salivarius, L. plantarum, L. paracasei, L. casei, L. brevis, B. breve, B. lactis, Bacillus subtilis, Bacillus licheniformis, and Saccaromyces boulardii.

13. The composition according to claim 1 where the pharmaceutically acceptable carrier is milk or milk portions.

14. The method according to claim 8, wherein said composition further comprises one or more species of bacteria selected from the genus Lactobacillus including Lactobacillus acidophilus, Lactobacillus rhamnosus, Lactobacillus casei, Lactobacillus gasseri, Lactobacillus fermentum, Lactobacillus amylolyticus, Lactobacillus casei subs. pseudoplantarum, Lactobacillus brevis, Lactobacillus salivarius, Lactobacillus plantarum, Lactobacillus fermentum, Lactobacillus jensenii, Lactobacillus crispatus, Lactobacillus vaginalis, Lactobacillus mucosae, Lactobacillus paracasei, and Lactobacillus coleohominis.

15. The method of claim 8, wherein said composition is applied directly to the rectum of a human as a suppository with use of an applicator.
